(19) United States
(12) Patent Application Publication
(10) Pub. No.: US 2023/0037213 A1
(43) Pub. Date: Feb. 2, 2023

(54) CHROMOSOME SEGMENT DERIVED FROM GOSSYPIUM ANOMALUM (G. ANOMALUM) LEADING TO LETHAL PHENOTYPE IN GOSSYPIUM HIRSUTUM (G. HIRSUTUM), AND MOLECULAR MARKERS THEREOF

(71) Applicant: JIANGSU ACADEMY OF AGRICULTURAL SCIENCES, Nanjing (CN)

(72) Inventors: Peng XU, Nanjing (CN); Xinlian SHEN, Nanjing (CN); Qi GUO, Nanjing (CN); Zhenzhen XU, Nanjing (CN); Shan MENG, Nanjing (CN)

(73) Assignee: JIANGSU ACADEMY OF AGRICULTURAL SCIENCES, Nanjing (CN)

(21) Appl. No.: 17/783,664

(22) PCT Filed: Jun. 2, 2021
(86) PCT No.: PCT/CN2021/097861

§ 371 Date: Jun. 9, 2022

(30) Foreign Application Priority Data
Jun. 24, 2020 (CN) 202010594408.1

(51) Int. Cl. C12Q 1/6895

(52) U.S. Cl. CPC C12Q 1/6895; C12Q 2600/13; C12Q 2600/156

(57) ABSTRACT

A chromosome segment derived from G. anomalum leading to a lethal phenotype in G. hirsutum, and molecular markers thereof are provided. The chromosome segment A11-9 is derived from G. anomalum, is located on chromosome 11 of a G. anomalum genome, and is marked by 6 pairs of simple sequence repeat (SSR) markers: NAU5192, A11_175, JAAS3191, A11_243, JAAS3310, and A11_1193. With DNA of G. anomalum as a template, the 6 pairs of SSR markers are used together to amplify the DNA of G. anomalum, and a chromosome segment with target fragments of the 6 pairs of SSR markers is the G. anomalum chromosome segment A11-9. A single chromosome segment introgression line derived from G. anomalum with a lethal phenotype is obtained, and the development of the single chromosome segment introgression line provides an important material for promoting the fine mapping of a target gene and the subsequent map-based cloning.

### CROSS REFERENCE TO THE RELATED APPLICATIONS

This application is the national phase entry of International Application No. PCT/CN2021/097861, filed on Jun. 2, 2021, which is based upon and claims priority to Chinese Patent Application No. 202010594408.1, filed on Jun. 24, 2020, the entire contents of which are incorporated herein by reference.

### SEQUENCE LISTING

The instant application contains a Sequence Listing which has been submitted in ASCII formatvia EFS-Web and is hereby incorporated by reference in its entirety. Said ASCII copy is named GBXRHD002-PKG_SEQUENCE_LISTING.txt, created on Mar. 17, 2022 and is 2,825 bytes in size.

### TECHNICAL FIELD

The present disclosure belongs to the technical field of molecular breeding, and specifically relates to a chromosome segment derived from Gossypium anomalum (G. anomalum) leading to a lethal phenotype in G. hirsutum and molecular markers thereof.

### BACKGROUND

As early as the early 20th century, it was found that wild resources could be used to compensate for the excellent alleles lost in modern breeding. However, interspecific reproductive isolation limits the use of wild resources in breeding. There are mainly three types of reproductive isolation. The first type thereof is geographic isolation, which indicates that creatures of the same kind are divided into different populations due to geographic barriers, such that gene flow fails to occur among the populations. The second type thereof is reproductive isolation before fertilization, which indicates that a hybrid zygote cannot be formed because species have different flowering times, or a pollen tube cannot germinate or cannot completely pass through a style and reach an ovary after a plant is pollinated, or male and female gametes are incompatible. The third type thereof is reproductive isolation after fertilization, which includes hybrid lethality (also referred to as hybrid weakness) and hybrid breakdown. Hybrid lethality or hybrid weakness occurs in the F₁ generation, showing plant lethality, sterility, shortness, and reduced viability. Some interspecific hybridization can produce normal hybrid F₁, but F₂ generation plants thereof often show weakness or lethality, which is referred to as hybrid breakdown. As a harmful genetic material, lethal genes are easily ignored and eliminated by breeders in production. However, special functions and active mechanisms of lethal genes endow lethal genes with significance in scientific research and production. Clarifying a mechanism of reproductive isolation can help to break reproductive barriers for traditional crop breeding and promote the interspecific genome exchange, which will facilitate the application of excellent traits of wild species in modern breeding.

Hybrid lethality and hybrid breakdown have been reported in different plants, which have simple inheritance modes. The hybrid lethality phenotype occurs in the F₁ generation, and is mostly controlled by one or two pairs of dominant genes. The hybrid breakdown phenotype occurs in the F₂ generation, and is mostly controlled by one or two pairs of recessive genes. The Bateson-Dobzhansky-Muller (BDM) model is proposed to explain the genetic basis of hybrid lethality and hybrid breakdown. The classic BDM model includes the epistatic interaction of two loci Each of branches derived from the common ancestor can form different new loci during an evolution process, and these loci are harmless in their respective genomes. However, in hybrids, these evolved genes interact, where when a new mutant gene is dominant, the F₁ generation shows hybrid lethality, and when a new mutant gene is recessive, the F₂ generation shows hybrid breakdown. The dominant BDM interaction pattern can also occur between single loci. An uncoordinated evolutionary process of a single locus shows that a gene locus of an ancestor evolves in different directions in different branches so as to form different genes in different populations. In the evolutionary process, an ancestral gene is coordinated with a gene formed after evolution. There is no fundamental difference between the single-locus interaction mode and the two-locus interaction mode. These genes evolve independently during an evolutionary process, and these loci have no negative effects in their respective genomes. Most reports of hybrid lethality and hybrid breakdown (Bomblies et al., 2007; Yamamoto et al., 2007; Chen et al., 2013, and Tikhenko et al., 2017) are results of action of the two-locus model There are few reports on the single-locus model (Smith et al., 2011).

The cloning of hybrid lethality genes of plants is a molecular basis for the analysis of hybrid lethality mechanism and is also a key to fundamentally interpreting the mechanism of hybrid lethality in plants, which helps to understand the importance of hybrid lethality gene evolution in reproductive isolation and speciation. In existing studies, genes related to hybrid lethality and hybrid breakdown have been subjected to genetic mapping in different species, and some genes have been cloned. With regard to the lethality molecular mechanism, it is generally believed that the hybrid lethality or hybrid breakdown mainly relates to an immune system of a plant. At present, the research on cotton hybrid lethality remains relatively rudimentary Many scholars have discovered the phenomenon of cotton hybrid lethality, and some lethal genes are mapped on chromosomes. However, there is no research report on molecular mechanism of cotton hybrid lethality.

### SUMMARY

One objective of the present disclosure is to provide a chromosome segment A11-9 derived from G. anomalum, which can cause a lethal phenotype in G. hirsutum.

Another objective of the present disclosure is to provide molecular markers of the G. anomalum chromosome segment A11-9 and primer sequences thereof, and the molecular markers are closely linked to the lethal phenotype derived from a G. anomalum chromosome segment introgression line.

The present disclosure also discloses the application of the molecular markers of the G. anomalum chromosome segment A11-9 and the primers thereof.

To solve the above technical problems, the present disclosure adopts the following technical solutions:

The present disclosure discloses a chromosome segment A11-9 derived from G. anomalum that could lead a lethal phenotype in G. hirsutum. The chromosome segment A11-9 is derived from G. anomalum, is located on chromosome 11 of the cotton genome, and is marked by 6 pairs of simple sequence repeat (SSR) markers: NAU5192, A11_75, JAAS3191, A11_243, JAAS3310, and A11_193, with DNA of G. anomalum as a template, the 6 pairs of SSR markers are used together to amplify the DNA of G. anomalum, and a chromosome segment with target size of fragments of the 6 SSR markers is the G. anomalum chromosome segment A11-9; and primer sequences and sizes of corresponding amplified fragments of the 6 SSR markers are as follows:

NAU5192: a forward primer sequence: SEQ ID NO. 1, a reverse primer sequence: SEQ ID NO. 2, and a size of the amplified target fragment: 280 bp;

A11_1175: a forward primer sequence: SEQ ID NO. 3, a reverse primer sequence: SEQ ID NO. 4, and a size of an amplified target fragment 210 bp;

JAAS3191: a forward primer sequence: SEQ ID NO. 5, a reverse primer sequence: SEQ ID NO. 6, and a size of an amplified target fragment: 270 bp;

A11_243: a forward primer sequence: SEQ ID NO. 7, a reverse primer sequence: SEQ ID NO. 8, and a size of an amplified target fragment. 280 bp;

JAAS3310: a forward primer sequence: SEQ ID NO. 9, a reverse primer sequence: SEQ ID NO. 10, and a size of an amplified target fragment: 250 bp; and

A11_193: a forward primer sequence: SEQ ID NO. 11, a reverse primer sequence: SEQ ID NO. 12, and a size of an amplified target fragment 250 bp.

The present disclosure also discloses molecular markers of the G. anomalum chromosome segment A11-9, where the molecular markers are NAU5192, A11_175, JAAS3191, A11_243, JAAS3310, and A11_193; and primer sequences and sizes of corresponding amplified fragments of the 6 SSR markers are as follows:

NAU5192: a forward primer sequence: SEQ ID NO. 1, a reverse primer sequence: SEQ ID NO. 2, and a size of an amplified target fragment in a G. anomalum genome: 280 bp;

A11_1175: a forward primer sequence: SEQ ID NO. 3, a reverse primer sequence: SEQ ID NO. 4, and a size of an amplified target fragment in the G. anomalum genome: 210 bp;

JAAS3191: a forward primer sequence: SEQ ID NO. 5, a reverse primer sequence: SEQ ID NO. 6, and a size of an amplified target fragment in the G. anomalum genome: 270 bp;

A11_243: a forward primer sequence: SEQ ID NO. 7, a reverse primer sequence: SEQ ID NO. 8, and a size of an amplified target fragment in the G. anomalum genome 280 bp;

JAAS3310: a forward primer sequence SEQ ID NO. 9, a reverse primer sequence: SEQ ID NO. 10, and a size of an amplified target fragment in the G. anomalum genome: 250 bp; and

A11_193: a forward primer sequence. SEQ ID NO. 11, a reverse primer sequence: SEQ ID NO. 12, and a size of an amplified target fragment in the G. anomalum genome: 250 bp.

In addition, the present disclosure discloses primers of SSR markers on the G. anomalum chromosome segment A11-9, where sequences of the primers are as follows:

SSR marker NAU5192: a forward primer sequence: SEQ ID NO. 1, and a reverse primer sequence: SEQ ID NO. 2;

SSR marker A11_175: a forward primer sequence: SEQ ID NO. 3, and a reverse primer sequence: SEQ ID NO. 4;

SSR marker JAAS3191: a forward primer sequence: SEQ ID NO. 5, and a reverse primer sequence: SEQ ID NO. 6;

SSR marker A11_243: a forward primer sequence: SEQ ID NO. 7, and a reverse primer sequence: SEQ ID NO. 8;

SSR marker JAAS3310: a forward primer sequence. SEQ ID NO. 9, and a reverse primer sequence: SEQ ID NO. 10; and

SSR marker A11_193: a forward primer sequence: SEQ ID NO. 11, and a reverse primer sequence: SEQ ID NO. 12.

In addition, the present disclosure also discloses use of the molecular markers or the primers for molecular markers described above in the genetic mapping of a lethal gene derived from G. anomalum and the identification of a lethal phenotype in cotton. The use described above can be conducted in accordance with a conventional method.

In addition, the present disclosure provides a kit including the primers of SSR markers, which can be used to determine whether a Gossypium material has a lethal phenotype. Furthermore, the present disclosure provides use of a reagent for detecting whether there are the molecular markers in the genetic mapping of a Gossypium lethal gene. The molecular markers of the present disclosure can be used for the genetic mapping of a Gossypium lethal gene. The use described above can be conducted in accordance with a conventional method.

A method for constructing a G. anomalum chromosome segment introgression line population used in the present disclosure includes the following steps:

(1) With G. hirsutum 86-1 as a female parent and G. anomalum as a male parent, chromosome doubling is conducted with colchicine to obtain hexaploid F₁ hybrids. Morphology, cytology, molecular marker, and other techniques are used to identify the obtained hexaploid hybrids with doubled status (Zhang et al., 2014). With hexaploid F₁ as a female parent and Su 8289 as a recurrent parent, continuous backcross was conducted four times. Through marker assisted selection (MAS), a G. anomalum chromosome segment introgression line population were obtained, where a single chromosome segment introgression line CSSL11-9 on chromosome A11 showed a lethal phenotype.

(2) The BC₄F₂ individuals of CSSL11-9 are panted, and the fresh leaves of CSSL11-9 are collected to extract DNA by the CTAB method; and then with the DNA as a template, the 6 pairs of SSR markers are used to conduct polymerase chain reaction (PCR) amplification. The sizes of the specific fragments amplified in the G. anomalum genome of the 6 SSR markers were 280 bp, 210 bp, 270 bp, 280 bp, 250 bp, and 250 bp, respectively. A chromosome segment with the specific fragments of the 6 molecular markers is the G. anomalum chromosome segment A11-9, and the BC₄F₂ individual with the G. anomalum chromosome segment A11-9 is the single chromosome segment introgression line CSSL11-9. Only a plant with homozygous G. anomalum molecular marker-specific fragments shows a lethal phenotype.

(3) Phenotypic identification of the lethal phenotype is conducted on individual plants every two weeks from the seedling stage to the mature stage in the field. The phenotypic traits are divided into a lethal phenotype and a normal phenotype. The lethal phenotype is specifically as follows: when a plant grows to having about 7 to 8 fruit-bearing shoots, top leaves first turn red, then other leaves throughout the plant gradually turn red, and finally all leaves wither and fall off, during which a top of the plant is necrotic and finally the plant is bare; and in a few cases, after the top of the plant is necrotic, two new lateral shoots sprout, which can bloom and undergo boll formation and opening normally, but have few bolls. Individual plants with leaves red on both front and back sides and lethal symptoms are classified as the lethal phenotype, while individual plants with normal green leaves throughout the plant are classified as the normal phenotype.

The present disclosure has the following advantages:

In the present disclosure, a single chromosome segment introgression line derived from G. anomalum with a lethal phenotype is obtained, and the development of the single chromosome segment introgression line provides an important material for promoting the fine mapping of a target gene and the subsequent map-based cloning.

The molecular markers of the present disclosure can lay a technical foundation for the study on the mechanisms of hybrid lethality, and the molecular markers can be used to quickly identify whether a Gossypium plant has a lethal phenotype. The molecular markers of the present disclosure have the characteristics of convenient detection, stable amplification product, and high specificity, and can be used to identify Gossypium materials simply and quickly with high throughput.

### BRIEF DESCRIPTION OF THE DRAWINGS

FIG. 1 shows target fragments amplified in Su 8289, CSSL11-9, and G. anomalum genomes using the 6 pairs of molecular markers on the G. anomalum chromosome segment A11-9 according to the present disclosure,

where lanes 1, 2, and 3 represent Su 8289, CSSL11-9, and G. anomalum, respectively, and the arrows indicate target fragments amplified specifically in G. anomalum.

FIGS. 2A-2D show field phenotypes of a plant with a normal phenotype and a plant with a lethal phenotype of the present disclosure,

where FIG. 2A and FIG. 2C show the plant with the normal phenotype, and FIG. 2B and FIG. 2D show the plant with the lethal phenotype.

### DETAILED DESCRIPTION OF THE EMBODIMENTS

The present disclosure will be described in detail below in conjunction with particular examples. The examples are provided to enable a more thorough understanding of the present disclosure and to fully convey the scope of the present disclosure to those skilled in the art.

The term “comprise” or “include” as used throughout the specification and claims is an open term and should be interpreted as “including but not limited to”. The subsequent description of the specification is a preferred implementation of the present disclosure. However, the description is based on the general principles of the specification and is not intended to limit the scope of the present disclosure. The protection scope of the present disclosure is defined by the appended claims

1. Experiment Materials

The G. hirsutum Su 8289 (recurrent parent) and G. anomalum (donor parent) used in the experiment were introduced by the Jiangsu Academy of Agricultural Sciences, which were recorded in “Caijiao Zhai, Peng Xu, Xia Zhang et al Development of Gossypium anomalum derived microsatellite markers and their use for genome-wide identification of recombination between the G. anomalum and G. hirsutum genomes. Theoretical and Applied Genetics, 2015, 128 (8): 1531-1540”. The public can obtain the materials from the applicants of the present disclosure, but the materials can only be used to repeat the experiment of the present disclosure, and cannot be used for other purposes. The materials can also be obtained through purchase

2. Experimental Method

2.1 Genomic DNA (gDNA) Extraction Method

CSSL11-9, Su 8289, and G. anomalum seeds were sown to obtain plants, and then gDNA was extracted A method for extracting the gDNA was as follows:

(1) 5 g of frozen leaves (or fresh leaves) were placed in a pre-cooled mortar, then liquid nitrogen was added, and the leaves were ground. 10 ml of a freshly prepared extraction buffer (Table 1) was added in twice, and a resulting mixture was then transferred to a 50 ml centrifuge tube, vortexed for thorough mixing, and stored in an ice bath for 10 min A resulting mixture was centrifuged at 4,000 rpm for 20 min (4° C.), and a resulting supernatant was discarded.

(2) 15 ml of a lysis buffer (Table 2) preheated at 65° C. was added to a resulting precipitate, and a resulting mixture was first stirred with a copper wire, then thoroughly vortexed, and incubated in a 65° C. water bath for 30 min.

(3) 15 ml of a mixed solution of chloroform and isoamyl alcohol (IAA) (in a volume ratio of 24.1) was added, and then the centrifuge tube was inverted 50 times or more and centrifuged at 4,000 rpm for 20 min (15° C.); a resulting supernatant was transferred to a 50 ml centrifuge tube, then pre-cooled isopropyl alcohol (IPA) was added at a volume 0.6 time a volume of the supernatant, and the centrifuge tube was slowly inverted 30 times for thorough mixing, allowed to stand for 10 min, and centrifuged at 4,000 rpm for 10 min (room temperature), a resulting supernatant was discarded, and 2 ml of 70% ethanol was added to a resulting precipitate for washing; and a resulting mixture was transferred to a 10 mi centrifuge tube and centrifuged at 10,000 rpm for 5 min, a resulting supernatant was discarded, and a resulting precipitate was air-dried for 20 min.

(4) 3 ml of a TE buffer (pH=8.0, 2.5 ml of a 1.0 M Tris-HCl solution and 0.5 ml of a 0.5 M ethylenediaminetetraacetic acid (EDTA) solution were thoroughly mixed, and a resulting solution was diluted with distilled water to 250 ml and then sterilized to obtain the TE buffer) was added for dissolution, a resulting mixture was incubated at 65° C. for 10 min to 30 min, an equal volume of a mixed solution of chloroform and IAA (in a volume ratio of 24:1) was added, and then the centrifuge tube was slowly inverted 50 times for thorough mixing, allowed to stand at room temperature for 5 min, and centrifuged at 10,000 rpm for 10 min.

(5) A resulting supernatant was transferred to a 10 ml centrifuge tube, 3 M sodium acetate (pH 5.2) was added at a volume 0.1 time a volume of the supernatant, and then an equal volume of IPA was added; the centrifuge tube was inverted 30 times, allowed to stand for 30 min, and centrifuged at 10,000 rpm for 5 min, a resulting supernatant was discarded, and 2 ml of 70% ethanol was added to wash a resulting DNA precipitate; and a resulting mixture was centrifuged at 10,000 rpm for 5 min, a resulting supernatant was discarded, and a resulting precipitate was air-dried for 20 min.

(6) 3 ml of a TE buffer was added for dissolution, and a resulting mixture was incubated at 65° C. for 10 min to 30 min.

(7) 5 μl of RNAase A (10 mg/ml) was added, and a resulting mixture was incubated at 37° C. for 30 min to 60 min; and an equal volume of a mixed solution of chloroform and IAA (in a volume ratio of 24:1) was added, and the centrifuge tube was slowly inverted 50 times for thorough mixing, allowed to stand at room temperature for 5 min, and centrifuged at 10,000 rpm for 10 min.

(8) A resulting supernatant was transferred to a 10 ml centrifuge tube. 3 M sodium acetate (pH 5.2) was added at a volume 0.1 time a volume of the supernatant, and then an equal volume of IRA was added; and the centrifuge tube was inverted 30 tines.

(9) A flocculent precipitate was collected into a 1.5 ml centrifuge Lube with 800 μl of 70% ethanol, and then the centrifuge tube was centrifuged at 10,000 rpm for 5 min; and a resulting supernatant was discarded, and a resulting DNA precipitate was air-dried.

(10) 200 μl of a TE buffer (10 mM Tris/HCl (pH 8.0), 1 mM EDTA (pH 8.0)] was added to dissolve DNA (4′C, 1 d to 2 d), and a resulting solution was stored at −20° C.

(11) 5 μl of 50 bp DNA Ladder (Takara Biotechnology (Dalian) Co., Ltd.) was used as a control, the DNA was serially diluted 5 times, 10 times, and 20 times, and the concentration, purity, and integrity of DNA were determined through 1% agarose gel electrophoresis.

(12) According to the concentration of extracted DNA, the DNA was diluted with TE, to obtain a 20 ng/dl working solution, which was thoroughly mixed for later use.

TABLE 1
DNA extraction buffer formula

| Working solution | ml | Stock solution |
|---|---|---|
| Glucose | 6.936 g | |
| 0.1 M Tris.HCl | 10.0 | 1.0 M Tris.HCl(pH 8.0) |
| 5 mM Na.EDTA | 1.0 | 0.5 M EDTA(pH 8.0) |
| 2% PVP | 20.0 | |
| 1% (V/V)β-ME | 1.0 | |
| dd Water | 68.0 | |
| Total | 100 ml | |

TABLE 2
Lysis buffer formula

| Working solution | ml | Stock solution |
|---|---|---|
| NaCl | 8.1816 g | |
| 0.1 M Tris.HCl | 10 | 1.0 M Tris.HCl(pH 8.0) |
| 20 mM Na.EDTA | 4 | 0.5 M Na.EDTA(pH 8.0) |
| 2% CTAB | 20 | |
| 2% PVP | 20 | |
| 1% (V/V)β-ME | 1.0 | |
| dd Water | 45 | |
| Total | 100 ml | |

2.2 PCR Method

(1) Reagents and Main Instruments Used

Taq enzyme and dNTPs used for PCR were purchased from Takara Biotechnology (Dalian) Co., Ltd. Reagents used for polyacrylamide gel electrophoresis (PAGE) included acrylamide, methylene bisacrylamide (MBA), Tris-base, boric acid, silver nitrate, sodium hydroxide, tetramethylethylenediamine (TEMED), and the like, which were purchased from Rongshengda Experimental Instrument Co., Ltd. The main instruments included Applied Biosystems PCR machine, Eppendor high-speed refrigerated centrifuge, water bath kettle, shaker, and electrophoresis tank and electrophoresis apparatus produced by Beijing Liuyi Instrument Factory.

(2) PCR System and Procedure

The PCR system was shown in Table 3.

TABLE 3
PCR reaction system

| Component | Volume |
|---|---|
| 10x buffer | 1.0 μL |
| 2.5 mM MgCl₂ | 1.0 μL |
| 10 mM dNTP_{S} | 0.2 μL |
| Primer R (5 μM/μL) | 1.0 μL |
| Primer F (5 μM/μL) | 1.0 μL |
| Taqase (2.5 U/μL) | 0.1 μL |
| Template DNA (20-100 ng) | 1.0 μL |
| ddH₂O | 4.7 μL |
| Total volume | 10.0 μL |

The PCR was conducted on the Applied Biosystems PCR machine, and a reaction procedure was as follows:

| | |
|---|---|
| 95° C. | 5 min |

| | | | |
|---|---|---|---|
| 94° C. | 30 s | | |
| 57° C. | 30 s | {close oversize brace} | 36cycles |
| 72° C. | 1 min | | |

| | |
|---|---|
| 72° C. | 10 min |
| 25° C. | For ever |

Electrophoresis Solution Preparation:

An amplification product was subjected to non-denaturing PAGE: gel concentration: 9%; electrophoresis buffer 0.5×TBE, and constant-voltage electrophoresis at 180 V for 1.5 h to 2 h.

9% PAGE gel: 43.5 g of acrylamide, 1.5 g of MBA, and 100 ml of 5×TBE were mixed, and a resulting mixture was diluted with distilled water to 500 ml and stored at 4° C.

10% ammonium persulfate (APS): 10 g of APS was dissolved in 100 ml of distilled water, and a resulting solution was stored at 4° C.

Loading buffer: 0.25 g of bromophenol blue (BPB), 0.25 g of xylene cyanol (XC), and 40 g of sucrose were mixed, and a resulting mixture was diluted with distilled water to 100 ml.

5×TBE: 54 g of Tris-base, 27.5 g of boric acid, and 20 ml of 0.5 M EDTA (PH=8.0) were mixed, and a resulting mixture was diluted with distilled water to 1 L and stored at room temperature.

Staining solution: 1 g of silver nitrate and 500 ml of distilled water were mixed.

Chromogenic solution. 7.5 g of sodium hydroxide, 750 μL of formaldehyde, and 500 ml of distilled water were mixed

Gel Preparation and Electrophoresis Process:

(1) Glass plates, gel strips, and combs were washed with clean water, and then air-dried for later use.

(2) The glass plates, gel strips, and combs were arranged as required, the bottom was covered with 1% agarose gel, and after the gel was solidified, these components were fixed on electrophoresis tanks.

(3) The prepared 9% PAGE gel was poured into a conical flask, and 10% APS and TEMED were added. The gel was poured quickly. After the conical flask was full, a comb was inserted; and 15 min to 20 min later, the comb was pulled out to be ready for electrophoresis.

(4) Before electrophoresis, 2 μL of the loading buffer was added to a PCR amplification product, then the comb was removed, and the electrophoresis buffer (1×TBE) was added to positive and negative electrophoresis tanks. A height of the buffer exceeded a short glass plate. 2 μL was loaded into each spot hole, and then constant-voltage electrophoresis was conducted at 180 V for 1.5 h to 2 h. When the blue indication mark was 2 cm below the gel, the electrophoresis was stopped, then the glass plates were carefully disassembled to remove the gel, and the gel was marked.

Staining and Chromogenic Process:

(1) Fixation: The removed gel was placed in a fixing solution (10% ethanol+0.5% glacial acetic acid) for 12 min.

(2) Staining: After the fixation was completed, the fixing solution was poured out, the staining solution (0.2% silver nitrate aqueous solution) was poured to conduct staining for 12 min, and the gel was rinsed with distilled water 3 times.

(3) Chromogenic reaction: 1.5% sodium hydroxide and 0.4% formaldehyde were added, and a resulting mixture was shaken until strips on a film were clearly displayed; and then the chromogenic solution was poured out, and the film was rinsed with tap water 4 times and photographed in a light box.

2.3 Selection and Identification of Markers

(1) With G. hirsutum 86-1 as a female parent and G. anomalum as a male parent, chromosome doubling was conducted with colchicine to obtain hexaploid F₁ hybrids.

Morphology, cytology, molecular marker, and other techniques were used to identify the obtained hexaploid hybrids with doubled status (Zhang et al., 2014). With hexaploid F₁ as a female parent and Su 8289 as a recurrent parent, continuous backcross was conducted four times. Through MAS, a G. anomalum chromosome segment introgression line population was obtained, where a single chromosome segment introgression line CSSL11-9 on chromosome A11 showed a lethal phenotype.

(2) Fresh leaves were collected from each of BC₄F₂ plants of the single chromosome segment introgression line CSSL11-9, the recurrent parent Su 8289, and G. anomalum, and then DNA was extracted by the CTAB method; and then with the DNA as a template, 230 pairs of SSR primers evenly covering the G. anomalum genome developed in this research group were used to identify the genome-wide foreground and background of CSSL11-9. The amplification products of the 6 pairs of SSR molecular marker primers in Table 4 were polymorphism between CSSL11-9 and the recurrent parent Su 8289. Then fresh leaves were collected to extract DNA by the CTAB method; and then with the DNA as a template, the 6 pairs of SSR markers were used as primers to conduct PCR amplification. A chromosome segment with the specific fragments of the 6 molecular markers was the G. anomalum chromosome segment A11-9, and the BC₄F₂ individual with the G. anomalum chromosome segment A11-9 is the single chromosome segment introgression line CSSL11-9. Only a plant with homozygous G. anomalum molecular marker-specific fragments showed a lethal phenotype.

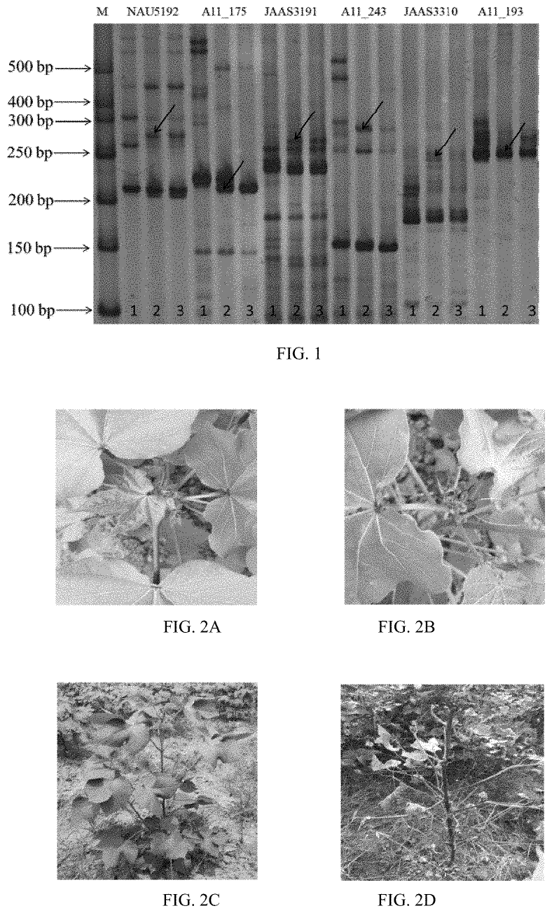

The specific fragments amplified in G. anomalum genome using the 6 molecular markers (NAU5192, A11_175, JAAS3191, A11_243, JAAS3310, and A11_193) had sizes of 280 bp, 210 bp, 270 bp, 280 bp, 250 bp, and 250 bp, respectively (FIG. 1). The 6 SSR markers were located on chromosome 11 of the cotton genome. Individual plants with all of the target fragments were selected (see Table 4) Through marker identification, it was determined that target fragments of the 6 molecular markers could be amplified in BC₄F₂ individual plants, and only plants with homozygous G. anomalum molecular marker-specific fragments showed a lethal phenotype, that is, CSSL11-9 with a lethal phenotype included the G. anomalum chromosome segment A11-9.

TABLE 4
SSR molecular markers on the chromosome segment A11-9 and sequences thereof

| | Fragment | | |
|---|---|---|---|
| Primer pair | size (bp) | Forward primer sequence 5′-3′ | Reverse primer sequence 5′-3′ |
| NAU5192 | 280 | ACAATGGAAGAAAAGCCTTG | TTCTTGCTCTGTTTCCCTTT |
| | | (SEQ ID NO. 1) | (SEQ ID NO. 2) |
| A11_175 | 210 | ACACCTATGCGCCAATGGAT | ACCTTGCCTCTCCCCATTTC |
| | | (SEQ ID NO. 3) | (SEQ ID NO. 4) |
| JAAS3191 | 270 | AGTGGAAATGGTGATAAGGTGCT | GCAACCCCCAAAACCACATC |
| | | (SEQ ID NO. 5) | (SEQ ID NO. 6) |
| A11_243 | 280 | GCTGTCGAGTGAGGATTGCT | GAGCTTAACTCCGACCACCC |
| | | (SEQ ID NO. 7) | (SEQ ID NO. 8) |
| JAAS3310 | 250 | CCTGCACCGATCTGCCTTTA | CTGAGTTCGACCCAGTTTCCA |
| | | (SEQ ID NO. 9) | (SEQ ID NO. 10) |
| A11_193 | 250 | CCGCTCTTGGCATCTCCTAG | TCACGATTCTCCCTCTGCTT |
| | | (SEQ ID NO. 11) | (SEQ ID NO. 12) |

Then, with Su 8289 as a female parent and CSSL11-9 as a male parent, an F₂ population with 2,337 individual plants was constructed, and phenotypic identification was conducted on individual plants every two weeks from the seedling stage to the mature stage in the field. The phenotypic traits were divided into a lethal phenotype and a normal phenotype. Individual plants with leaves red on both front and back sides and lethal symptoms were classified as the lethal phenotype, while individual plants with normal green leaves throughout the plant were classified as the normal phenotype. Results showed that, among the 2,337 F₂ individual plants, 545 individual plants showed a lethal phenotype, and 1,792 individual plants showed a normal phenotype, χ_{c}²=3.516<χ_{c}²_{0.005.1}=3.84, indicating that the normal phenotype and the lethal phenotype conformed to the phenotypic ratio of 3:1. Therefore, it was believed that the lethal phenotype derived from G. anomalum was controlled by a pair of recessive genes in this population.

TABLE 5
Genotypes of different phenotype materials

| Material | Phenotype | NAU5172 | A11_175 | JAAS3191 | A11_243 | JAAS3310 | A11_193 |
|---|---|---|---|---|---|---|---|
| F₁ | Normal | 3 | 3 | 3 | 3 | 3 | 3 |
| Su 8289 | Normal | 1 | 1 | 1 | 1 | 1 | 1 |
| CSSL11-9 | Lethal | 2 | 2 | 2 | 2 | 2 | 2 |

1: Su 8269 genotype;
2: G. anomalum genotype; and
3: heterozygous genotype

2.4 Phenotypic Identification of the Lethal Phenotype

Phenotypic identification of the lethal phenotype was conducted on individual plants every two weeks from the seedling stage to the mature stage in the field. The phenotypic traits were divided into a lethal phenotype and a normal phenotype. The lethal trait was specifically as follows: when a plant grew to have about 7 to 8 fruit-bearing shoots, top leaves first turned red, then other leaves throughout the plant gradually turned red (FIG. 23), and finally all leaves withered and fell off, during which a top of the plant was necrotic and finally the plant was bare (FIG. 2D); and in a few cases, after the top of the plant was necrotic, two new lateral shoots sprouted, which could bloom and undergo boll formation and opening normally, but had few bolls Individual plants with leaves red on both front and back sides and lethal symptoms were classified as the lethal phenotype, while individual plants with normal green leaves throughout the plant were classified as the normal phenotype (FIG. 2A and FIG. 2C).

Although the present disclosure has been described in detail above with general descriptions and specific examples, it will be apparent to those skilled in the art that some modifications or improvements can be made on the basis of the present disclosure Therefore, all these modifications or improvements made without departing from the spirit of the present disclosure fall within the scope of the present disclosure.

What is claimed is:

1. A chromosome segment derived from Gossypium anomalum (G. anomalum) leading to a lethal phenotype in Gossypium hirsutum (G. hirsutum), wherein the chromosome segment A11-9 is derived from G. anomalum, is located on chromosome 11 of a G. anomalum genome, and is marked by 6 pairs of simple sequence repeat (SSR) markers comprising NAU5192, A11_175, JAAS3191, A11_243, JAAS3310, and A11_193: with DNA of the G. anomalum as a template, the 6 pairs of SSR markers are used together to amplify the DNA of the G. anomalum, and a chromosome segment with target fragments of the 6 pairs of SSR markers is the G. anomalum chromosome segment A11-9; and primer sequences of the 6 SSR markers and sizes of corresponding amplified fragments are as follows:
NAU5192: a forward primer sequence: SEQ ID NO. 1, a reverse primer sequence: SEQ ID NO. 2, and a size of an amplified target fragment: 280 bp;
A11_175: a forward primer sequence: SEQ ID NO. 3, a reverse primer sequence: SEQ ID NO. 4, and a size of an amplified target fragment: 210 bp;
JAAS3191: a forward primer sequence: SEQ ID NO. 5, a reverse primer sequence: SEQ ID NO 6, and a size of an amplified target fragment: 270 bp;
A11_243: a forward primer sequence: SEQ ID NO. 7, a reverse primer sequence: SEQ ID NO. 8, and a size of an amplified target fragment: 280 bp;
JAAS3310: a forward primer sequence: SEQ ID NO. 9, a reverse primer sequence: SEQ ID NO. 10, and a size of an amplified target fragment: 250 bp; and
A11_193: a forward primer sequence: SEQ ID NO. 11, a reverse primer sequence: SEQ ID NO. 12, and a size of an amplified target fragment: 250 bp.

2. Molecular markers of a G. anomalum chromosome segment A11-9, wherein the molecular markers are composed of NAU5192, A11_175, JAAS3191, A11_243, JAAS3310, and A11_193; and primer sequences of the molecular markers and sizes of corresponding amplified target fragments are as follows:
NAU5192: a forward primer sequence: SEQ ID NO. 1, a reverse primer sequence: SEQ ID NO. 2, and a size of an amplified target fragment in a G. anomalum genome: 280 bp;
A11_1175: a forward primer sequence: SEQ ID NO. 3, a reverse primer sequence: SEQ ID NO. 4, and a size of an amplified target fragment in the G. anomalum genome: 210 bp;
JAAS3191: a forward primer sequence: SEQ ID NO. 5, a reverse primer sequence: SEQ ID NO. 6, and a size of an amplified target fragment in the G. anomalum genome: 270 bp;
A11_243: a forward primer sequence: SEQ ID NO. 7, a reverse primer sequence: SEQ ID NO 8, and a size of an amplified target fragment in the G. anomalum genome 280 bp;
JAAS3310: a forward primer sequence: SEQ ID NO. 9, a reverse primer sequence: SEQ ID NO. 10, and a size of an amplified target fragment in the G. anomalum genome: 250 bp; and
A11_193: a forward primer sequence: SEQ ID NO. 11, a reverse primer sequence: SEQ ID NO. 12, and a size of an amplified target fragment in the G. anomalum genome: 250 bp.

3. Primers for SSR markers on a G. anomalum chromosome segment A11-9, wherein sequences of the primers are as follows:
NAU5192: a forward primer sequence: SEQ ID NO. 1, and a reverse primer sequence: SEQ ID NO 2;
A11_175: a forward primer sequence: SEQ ID NO. 3, and a reverse primer sequence: SEQ ID NO. 4;
JAAS3191: a forward primer sequence: SEQ ID NO. 5, and a reverse primer sequence: SEQ ID NO. 6;
A11_243: a forward primer sequence: SEQ ID NO. 7, and a reverse primer sequence: SEQ ID NO. 8;
JAAS3310: a forward primer sequence: SEQ ID NO. 9, and a reverse primer sequence: SEQ ID NO 10; and
A11_193: a forward primer sequence: SEQ ID NO. 11, and a reverse primer sequence: SEQ ID NO. 12.

4. Use of the molecular markers according to claim 2 in G. anomalum lethal gene mapping and Gossypium molecular breeding.

5. The use according to claim 4, wherein the molecular markers are used to identify or assist in an identification of a lethal phenotype of a Gossypium plant.

6. The use according to claim 5, wherein the lethal phenotype is specifically as follows: when a plant grows to have 7 to 8 fruit-bearing shoots, top leaves first turn red, then other leaves throughout the plant gradually turn red, and finally all leaves wither and fall off, concurrently, a top of the plant is necrotic and finally the plant is bare; or
after the top of the plant is necrotic, two new lateral shoots sprout, the two new lateral shoots bloom and undergo boll formation and opening normally, but have few bolls.

7. A kit comprising the primers for the SSR markers according to claim 3.

8. Use of the kit according to claim 7 in an identification of a lethal phenotype of a Gossypium plant.

9. Use of a reagent for detecting whether there is the molecular markers according to claim 2 in a mapping of a Gossypium lethal gene and/or an identification of a Gossypium lethal phenotype.

10. Use of the primers for the SSR markers according to claim 3 in G. anomalum lethal gene mapping and Gossypium molecular breeding.


SEQUENCE LISTING (12 sequences)

SEQ ID NO: 1 - DNA, 20 bp, Artificial Sequence
  forward primer for NAU5192
acaatggaagaaaagccttg

SEQ ID NO: 2 - DNA, 20 bp, Artificial Sequence
  reverse primer for NAU5192
ttcttgctctgtttcccttt

SEQ ID NO: 3 - DNA, 20 bp, Artificial Sequence
  forward primer for A11_175
acacctatgcgccaatggat

SEQ ID NO: 4 - DNA, 20 bp, Artificial Sequence
  reverse primer for A11_175
accttgcctctccccatttc

SEQ ID NO: 5 - DNA, 23 bp, Artificial Sequence
  forward primer for JAAS3191
agtggaaatggtgataaggtgct

SEQ ID NO: 6 - DNA, 20 bp, Artificial Sequence
  reverse primer for JAAS3191
gcaacccccaaaaccacatc

SEQ ID NO: 7 - DNA, 20 bp, Artificial Sequence
  forward primer for A11_243
gctgtcgagtgaggattgct

SEQ ID NO: 8 - DNA, 20 bp, Artificial Sequence
  reverse primer for A11_243
gagcttaactccgaccaccc

SEQ ID NO: 9 - DNA, 20 bp, Artificial Sequence
  forward primer for JAAS3310
cctgcaccgatctgccttta

SEQ ID NO: 10 - DNA, 21 bp, Artificial Sequence
  reverse primer for JAAS3310
ctgagttcgacccagtttcca

SEQ ID NO: 11 - DNA, 20 bp, Artificial Sequence
  forward primer for A11_193
ccgctcttggcatctcctag

SEQ ID NO: 12 - DNA, 20 bp, Artificial Sequence
  reverse primer for A11_193
tcacgattctccctctgctt